(19) United States
(12) Patent Application Publication
(10) Pub. No.: US 2004/0115271 A1
(43) Pub. Date: Jun. 17, 2004

(54) Hydration compositions for corneal pre-surgery treatment

(76) Inventor: Alex Sacharoff, Oviedo, FL (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/473,651

(22) PCT Filed: Jun. 21, 2002
(86) PCT No.: PCT/US02/19784

(51) Int. Cl. A61K 9/14; A61K 31/715

(52) U.S. Cl. 424/486; 424/488; 514/54

(57) ABSTRACT

Compositions and methods for corneal tissue treatment prior to surgery are disclosed. It has been discovered that an important factor contributing to the variance between predicted and actual results in both photoablation and mechanical resection of corneal tissue is the degree of hydration of the tissue, particularly the degree of hydration in the surface layers of tissue. The compositions of the invention contain a polymeric matrix and a hydration fluid, the fluid being held in the matrix by a predefined osmotic pressure such that upon application of the composition to the corneal surface, a standardized level of hydration is achieved in the corneal tissue by fluid transfer between the matrix and the tissue. In another aspect of the invention, methods for pre-treating corneal tissue prior to surgery are disclosed involving the application of the compositions of the invention to the corneal tissue, such that a standardized level of hydration is achieved in the corneal tissue by fluid transfer between the matrix and the tissue; and then maintaining the composition in contact with the corneal tissue until a desired state of hydration in the tissue is achieved.

### BACKGROUND OF THE INVENTION

The technical field of this invention is corneal surgery and, in particular, the invention relates to compositions and methods for improving the predictability of laser vision correction procedures using ablative radiation and/or corneal keratectomies performed with mechanical instruments.

Recently, it has been demonstrated that changes in the refractive power of the eye can be achieved by laser ablation of the corneal surface. Such procedures, known as photorefractive keratectomy, involves the use of a nonthermal, high energy, laser radiation to sculpt the cornea into an ideal shape. For details, see, Marshall et al. “Photoablative Reprofiling of the Cornea using an Excimer Laser: Photorefractive Keratectomy,” Vol. 1, Lasers in Ophthalmology, pp. 2148 (1986); and Tuft et al. “Stromal Remodeling Following Photorefractive Keratectomy,” Vol. 1, Lasers in Ophthalmology, pp. 177-183 (1987), herein incorporated by reference.

The cornea of the eye comprises transparent avascular tissue. The cornea functions as both a protective, anterior membrane and a “window” through which light passes as it proceeds to the retina. The cornea is composed of a set of distinct layers: the outer epithelium, an anterior elastic lamina known as “Bowman's membrane,” the cornea proper (or “stroma”), a posterior elastic lamina known as “Descemet's membrane”, and the inner endothelium. The stroma is fibrous and constitutes the major portion of the cornea. Bowman's membrane, which forms the outer elastic lamina, is a rigid fibrillar structure not tending to cut or fracture, while Descemet's membrane, which forms the inner elastic lamina, is very brittle but elastic and has a tendency to curl. Together, the Bowman's and Descemet's membranes impart the necessary curvature to the stromal tissue. This curvature of the cornea constitutes an major component of the refractive power of the eye, thereby allowing objects to be imaged onto the retina.

The average adult cornea is about 0.65 mm thick at the periphery, and about 0.54 mm thick in the center. Photorefractive keratectomy involves sculpting the uppermost regions of the cornea, namely, the epithelium, Bowman's membrane, and/or the outer stroma The epithelium consists of five or six layers of cells, and the underlying Bowman's membrane, is also a very thin structure. The corneal stroma accounts for about 90 percent of the corneal thickness.

In photorefractive keratectomies (“laser vision correction”), a laser photoablation apparatus is used to change the curvature of the cornea, at least in the so-called “optical zone” or region of the cornea through which light must pass to enter the pupil and reach the retina. The size of the optical zone will, of course, vary from individual to individual, and will also vary based upon ambient light conditions (because the pupil will dilate and contract in response to ambient light). The extent of the sculpted region (and the depth of ablation) will depend on the amount of correction needed to achieve optimal vision. For example, correction of relatively mild myopia (nearsightedness) on the order of 2 Diopters requires only a modest flattening of the corneal curvature, which can be accomplished in a region of small cross-sectional area (e.g., affecting a circular region of the cornea in front of the pupil less than 5 millimeters in diameter). However, when more complicated refractive errors, such as more severe myopia, hyperopia (farsightedness) or astigmatisms, are corrected by photorefractive keratectomy procedures, the sculpted area will extend across a much larger portion of the cornea, e.g., affecting a region as large as 8 mm in diameter or more.

One approach to performing photorefractive keratectomy procedures is to employ an optical system which varies the size of the exposed surface area to which the laser radiation is applied. In one embodiment of such a “variable exposure area” system, an adjustable iris can be deposed in the beam path to increase or decrease the region of cornea on which the laser radiation is incident. By progressively varying the size of the exposed region, a desired photoablation profile is established on the surface. For further details on these techniques see U.S. Pat. No. 4,973,330 issued to Azema et al. on Nov. 27, 1990, and U.S. Pat. No. 4,941,093 issued to Marshall et al. on Jul. 10, 1990, the entire contents of which are incorporated herein by reference.

Another technique for corneal reshaping involves the use of a beam-shaping mask which is disposed between the laser and the surface. The mask provides a predefined profile of resistance to erosion by laser radiation selectively absorbing some of the laser radiation while permitting the remainder to be transmitted to the surface in accordance with the mask profile. For further disclosures of such masking techniques, see U.S. Pat. No. 4,856,513 issued to Muller on Aug. 15, 1989; U.S. Pat. No. 4,994,058 issued to Raven et al. on Feb. 19, 1991; U.S. Pat. No. 5,019,074 issued to Muller on May 28, 1991; and U.S. Pat. No. 5,324,281 issued to Muller on Jun. 28, 1994; the entire contents of the foregoing patents are incorporated herein by reference.

Yet another approach to laser vision correction relies upon a small mobile spot of ablative radiation which is “scanned” across the cornea in a predefined pattern to reshape the corneal curvature. The laser beam is either repeatedly scanned across the cornea or otherwise controlled to expose the cornea to differing amounts of ablative radiation over time so as to effect a cumulative reprofiling of the corneal surface. For a disclosure of scanning systems, see, for example, U.S. Pat. No. 5,980,513 issued to Frey et al. on Nov. 9, 1999, the entire contents of which are incorporated herein by reference.

In one commonly practiced type of laser vision correction procedure, known as Laser Assisted In Situ Keratoplasty (“LASIK”), a microkeratome is used to remove (or hingedly displace) an anterior lamina of the cornea. A laser is then used to selectively ablate stromal tissue, after which the anterior lamina is replaced. LASIK procedures are often preferred because the Bowman's membrane of the patient's eye remains intact, thus preserving its structural integrity and reducing the time necessary for healing. However, LASIK procedures require precision in the removal of the anterior lamina.

In another type of laser vision correction procedure, known as laser epithelial keratomileusis or “LASEK”, the epithelium sheet is loosened with an alcohol solution, then rolled back to expose the stroma. The excimer laser is then used to change the shape of the stroma and the loosened epithelial sheet is repositioned over the stroma.

In all of the above-mentioned laser vision correction procedures, precision and predictability from one patient to the next is essential. In both laser ablation and mechanical resections, the results can depend on several factors, including the physiological properties of the corneal tissue.

There exists a need for improved methods of corneal surgery, especially methods that can reduce the variability of results. In addition, compositions suitable for application prior to corneal surgery, which can reduce physiological differences in the patient population, would satisfy a long-felt need in the art.

### SUMMARY OF THE INVENTION

The present invention is directed to the provision of compositions and methods for achieving a uniform hydration of corneal tissue prior to laser vision correction procedures. It has been discovered that an important factor contributing to the variance between predicted and actual results in both photoablation and mechanical resection of corneal tissue is the degree of hydration of the tissue, particularly the degree of hydration in the surface layers of tissue.

The compositions of the invention contain a polymeric matrix and a hydration fluid, the fluid being held in the matrix by a predefined osmotic pressure such that upon application of the composition to the corneal surface, a standardized level of hydration is achieved in the corneal tissue by fluid transfer between the matrix and the tissue.

The compositions of the present invention are preferably sterilized, so as to insure that the compositions do not provide a source of infection when applied to the eye during vision correction procedures.

The compositions of the invention can further comprise polyphase systems and may contain non-aqueous solutes, non-aqueous solvents, and other non-aqueous additives. Homogeneous, polyphase systems can contain such additives as water insoluble, high molecular weight fatty acids and alcohols, fixed oils, volatile oils and waxes, mono-, di-, and triglycerides, and synthetic, water insoluble polymers without altering the functionality of the system.

The preferred hydrating compositions of the present invention comprise aqueous mixtures of a matrix-forming, water-soluble polymer and an ionic polysaccharide, optionally containing a latent counter-ion to gel the polysaccharide upon release of the counter-ion. Alternatively, the compositions of the invention can comprise two part aqueous gel systems, one part of which contains the ionic polysaccharide and matrix forming polymer and the other part containing an aqueous solution of a counter-ion.

The hydrating compositions of the invention can be removed just prior to surgery.

The useful matrix forming polymers are, preferably, water-soluble polymers such as those which have been used in ophthalmic applications. The hydroxyalkyl cellulosics and methyl celluloses, sodium hyaluronate, and polyvinyl alcohol are representative polymers which have been found useful in ophthalmic applications.

Useful ionic polysaccharides include natural polymers such as chitosan, gellan gum or alginates. Aqueous solutions of alginate ionic polysaccharides form gels upon contact with aqueous solutions of counter-ions such as calcium, strontium, aluminum, etc. Aqueous solutions of chitosan form gels upon contact with a metal tripolyphosphate counter-ion. In general, when ionic polysaccharides are present in aqueous solutions in admixture with matrix forming polymers and suitable counter-ions, such mixtures can form useful gels. The osmolality of which can be calculated by assuming that the matrix forming polymer, if water soluble, does not contribute to the osmolality in the gel state.

In another aspect of the invention, methods for pre-treating corneal tissue prior to surgery are disclosed involving the application of the compositions of the invention to the corneal tissue, such that a standardized level of hydration is achieved in the corneal tissue by fluid transfer between the matrix and the tissue; and then maintaining the composition in contact with the corneal tissue until a desired state of hydration in the tissue is achieved.

### DETAILED DESCRIPTION OF THE INVENTION

Representative useful matrix forming polymers are water soluble alkyl celluloses, such as methyl and ethyl cellulose; hydroxyalkyl celluloses, such as hydroxypropylmethyl cellulose and hydroxyethyl cellulose; hyaluronic acid and water soluble salts thereof such as sodium hyaluronate; chondroitin sulfate and water soluble salts thereof, such as sodium chondroitin sulfate; polymers of acrylamide, acrylic acid, and polycyanoacrylates; polymers of methyl methacrylate and 2-hydroxyethyl metbacrylate; polydextrose; cyclodextrin; polydextrin; maltodextrin, dextran; gelatin; collagen; natural gums, such as xanthan, locust bean, acacia, tragacanth, carrageenan, and agar; derivatives of polygalacturonic acid, such as pectin; polyvinyl alcohol; polyvinyl pyrrolidone; polyethylene glycol; and polyethylene oxide. One preferred matrix forming agent is carboxymethyl cellulose and its sodium salt. Further details regarding preferred water soluble, matrix forming polymers are provided below.

Cyclodextrin, also known as cycloamylose, is a cyclic oligosaccharide. Cyclodextrins are produced by the enzyme conversion of prehydrolized starch to a mixture of alpha, beta, and gamma cyclodextrins and some linear dextrins. The cyclodextrins are composed of glucose units linked together by alpha (14) glycosidic bonds.

Sodium hyaluronate, also known as hyaluronic acid, is composed of repeating units of sodium glucuronate and N-acetylglucosamine.

Polydextrose is a randomly bonded condensation polymer of dextrose which is only partially metabolized by mammals. The polymer can contain a minor amount of bound sorbitol, citric acid, and glucose.

Chondroitin sulfate, also known as sodium chondroitin sulfate, is a mucopolysaccharide found in every part of human tissue, specifically cartilage, bones, tendons, ligaments, and vascular walls. This polysaccharide has been extracted and purified from the cartilage of sharks.

Carrageenan is a linear polysaccharide having repeating galactose units and 3,6 anhydrogalactose units, both of which can be sulfated or nonsulfated, joined by alternating 1-3 and beta 1-4 glycosidic linkages. Carrageenan is a hydrocolloid which is heat extracted from several species of red seaweed and irish moss.

Maltodextrins are water soluble glucose polymers which are formed by the reaction of starch with an acid and/or enzymes in the presence of water.

Further details regarding the composition and derivation of other useful water soluble, matrix forming polymers can be found in the HANDBOOK OF PHARMACEUTICAL EXCIPIENTS, published by the American Pharmaceutical Association Washington, D.C. (1986), the entire contents of which are incorporated herein by reference.

A gel-forming, ionic polysaccharide may also be utilized as the polymeric matrix component of the ocular hydration compositions described herein, either alone or in combination with one or more of the matrix-forming polymers described above. The ionic polysaccharides useful in the present invention typically are hydrophilic colloidal materials and include natural gums, such as gellan gum, alginate gums and mixtures thereof.

The alginates can be any of the water-soluble alginates, including the alkali metal alginates, such as sodium, potassium, lithium, rubidium and cesium salts of alginic acid, as well as the ammonium salt, and the soluble alginates of an organic base such as mono-, di-, or tri-ethanolamine alginates, aniline alginates, and the like. Chitosan, which is the common name for deacetylated chitin, is also useful. Chitin is a natural product comprising poly-(N-acetyl-D-glucosamine). Gellan gum is produced from the fermentation of pseudomonas elodea to yield an extracellular heteropolysaccharide. The alginates and chitosan are available from Protan, Inc., Commack, N.Y., and gellan gum is available from the Kelco Division of Merck & Co., Inc., San Diego, Calif.

Any cross linking agent having more than one functional group wherein the function group is either chemical or ionic may be utilized to cross link the polysaccharides described above. As known in the art, cross linking can occur between molecules of similar polymers by physical reaction as long as appropriate functional groups are present on the polymers.

Useful counter-ions for gelling the gellan gum or alginate ionic polysaccharides in combination with the matrix forming, water soluble polymer compositions of the invention are cationic gelling agents, preferably, comprising a divalent or trivalent cation. Useful divalent cations include the alkaline earth metals, preferably, selected from the group consisting of calcium and strontium. Useful trivalent cations include aluminum. The most preferred counter-ions for gelling gellan gum or alginate ionic polysaccharides are contained in ionic compounds selected from pharmaceutically-acceptable gluconates, flourides, citrates, phosphates, tartrates, sulfates, acetates, borates, chlorides, and the like having alkaline earth metal cations such as calcium and strontium. Especially preferred counter-ion containing inorganic salts for use as ionic polysaccharide gelling agents include such inorganic salts as the chloride salts, such as strontium chloride, calcium chloride, and mixtures thereof. Generally, a molar ratio of counter-ion to gellan, chitosan or alginate of about 1:1 to about 10:1, preferably, about 2:1 to about 5:1, and, most preferably, about 3:1 to about 5:1 is used.

While the counter-ion, such as calcium or other counter-ions may be obtained by contact of the compositions of the invention with bodily fluids, it is preferred that a counter-ion in latent form be used in combination with the gellan gum or alginate ionic polysaccharide and matrix forming, water soluble polymer compositions of the invention. Alternatively, a counter-ion can be combined with the ionic polysaccharide and water soluble, matrix forming polymer compositions of the invention utilizing a two part system in which the counter-ion is topically or otherwise applied to the compositions of the invention subsequent to their topical or other application.

Incorporation of the counter-ion in a latent form together with the ionic polysaccharide and matrix forming, water soluble polymer compositions of the invention may be accomplished by either encapsulating an aqueous solution of one of the counter-ion gelling agents, previously described above, or by the incorporation of the counter-ion gelling agent into a matrix which provides for the controlled, slow-release of gelatin-encapsulated controlled release compositions, as described in U.S. Pat. No. 4,795,642 issued to Cohen, et al. on Jan. 3, 1989, the entire contents of which are incorporated herein by reference. The '642 patent describes the preparation of a gelatin shell encapsulating a controlled release formulation in which the gelatin composition includes calcium chloride as the gelling agent. Alternatively, the counter-ion can be the incorporated as an aqueous solution of a cationic gelling agent encapsulated in a vesical composed, for instance, of alpha-tocopherol, as described in U.S. Pat. No. 4,861,580 issued to Janoff, et al. on Aug. 29, 1989, the entire contents of which are incorporated herein by reference.

Generally, aqueous compositions comprising chitosan can be gelled with multivalent anion gelling agents, preferably, comprising a metal polyphosphate, such as an alkali metal or ammonium polyphosphates, pyrophosphates, or metaphosphates. Representative metaphosphate, pyrophosphate, and polyphosphate gelling agents include sodium and potassium, polyphosphates, sodium and potassium pyrophosphates, sodium and potassium metaphosphates, and sodium and ammonium (mono-, di-, tri-) phosphates.

The hydrating compositions of the present invention will contain the polymeric matrix in an amount of from about 1% to about 50% by weight, based on the total weight of the composition (“wt. %”). The amounts of gel forming ionic polysaccharide and water soluble, matrix forming polymer may be varied to increase or decrease the gelation properties of the compositions.

The compositions of the present invention also contain an ocular hydration fluid, such as water, a balanced salt solution, or other physiologically acceptable fluid that is capable of diffusing from the composition into the corneal tissue.

The pH and osmolality of the hydrating compositions described above must be compatible with the pH and osmolality of lachrymal fluids and corneal tissues. By matching the osmolality of the compositions to that of natural, healthy stromal tissue, it is possible to control inherent variability in patient corneal hydration. The preferred osmolality range is 250 to 350 milliosmoles/kilogram of water (“mOsm/kg”).

The ocular hydrating compositions of the present invention may also contain one or more drugs or diagnostic agents.

If desired, the compositions of the invention may also contain preservatives, cosolvents, suspending agents, viscosity enhancing agents, ionic-strength and osmolality adjustors and other excipients. However, the compositions preferably do not contain antimicrobial preservative agents, since such agents (e.g., benzalkonium chloride) can cause ocular irritation. The compositions of the present invention that do not contain antimicrobial preservative agents are referred to herein as being “unpreserved” or “preservative free”.

Suitable water soluble buffering agents are alkali or alkali earth carbonates, phosphates, bicarbonates, citrates, borates, acetates, succinates and the like, such as sodium phosphate, citrate, borate, acetate, bicarbonate, carbonate and tromethamine (TRIS). These agents are present in amounts sufficient to maintain the pH of the system at 7.4±0.2 and preferably, 7.4. As such the buffering agent can be as much as 5% on a weight basis of the total composition.

The hydrating compositions of the present invention can be prepared in accordance with the following procedures. A mixture of a water soluble, matrix forming polymer and ionic polysaccharide is stirred or shaken in admixture with an aqueous buffer solution to bring about a more rapid solution of the polymer. Additional hydrating fluid and various additives such as salts and preservatives can subsequently be added and dissolved. In some instances, pharmacologically active substances must be suspended if they are insoluble in water. A pH of 7.4+/−0.2 is obtained by of appropriate buffering agents.

1. A therapeutic composition for application to corneal tissue comprising:
a polymeric matrix and
a hydration fluid, the fluid being held in the matrix by a predefined osmotic pressure such that upon application of the composition to the corneal surface, a standardized level of hydration is achieved in the corneal tissue by fluid transfer between the matrix and the tissue.

2. The composition of claim 1 wherein the predefined osmotic pressure of the fluid in the matrix ranges from about 250 to about 350 mOsm/kg.

3. The composition of claim 1 wherein the polymeric matrix comprises a hydrogel.

4. The composition of claim 1 wherein the matrix further comprises at least one polymer selected from the group consisting of polysaccharides, carboxymethylcelluloses, alkyl methyl celluloses, hydroxyalkyl methyl celluloses, hyaluronic acid, sodium chondroitin sulfate, polyacrylic acid, polyacrylamide, polycyanolacrylates, alkyl methacrylatepolymers, hydroxyalkyl methacrylate polymers, cyclodextrin, polydextrose, dextran, gelatin, polygalacturonic acid, polyvinyl alcohol, polyvinyl pyrrolidone, polyalkylene glycols and polyethylene oxide.

5. The composition of claim 1 wherein the polymeric matrix comprises a polysaccharide.

6. The composition of claim 5 wherein the polysaccharide comprises gellan gum, alginate gum or chitosan.

7. The composition of claim 1 wherein the matrix comprises a carboxymethylcellulose.

8. The composition of claim 1 wherein said composition further comprises a drug selected from the group consisting of antibacterials, antihistamines, decongestants, antiinflammatories, antiparasitics, miotics, anticholinergies, antivirals, local anesthetics, antifungals, amoebicidals, trichomonocidals, analgesics, mydriatics, antiglaucoma drugs, carbonic anhydrase inhibitors, chelating agents, antineoplastics, antihypertensives, and muscle relaxants.

9. A method for pretreating corneal tissue prior to surgery comprising:
applying a composition to the corneal tissue, the composition comprising:
a polymeric matrix and
a hydration fluid, the fluid being held in the matrix by a predefined osmotic pressure such that upon application of the composition to the corneal surface, a standardized level of hydration is achieved in the corneal tissue by fluid transfer between the matrix and the tissue; and
maintaining the composition in contact with the corneal tissue until a desired state of hydration in the tissue is achieved.
